(19) United States
(12) Patent Application Publication
(10) Pub. No.: US 2023/0025943 A1
(43) Pub. Date: Jan. 26, 2023

(54) LOCATION-AWARE WELL-BEING INSIGHTS

(71) Applicant: Microsoft Technology Licensing, LLC, Redmond, WA (US)

(72) Inventor: Antonios KARATZOGLOU, Vancouver (CA)

(21) Appl. No.: 17/385,672

(22) Filed: Jul. 26, 2021

(51) Int. Cl. G16H 20/60; G16H 50/30; H04W 4/029

(52) U.S. Cl. CPC G16H 20/60; G16H 50/30; H04W 4/029

(57) ABSTRACT

The present disclosure relates to systems, devices, and methods for providing location-aware insights. The systems, devices, and methods may determine a visit history for a user that includes a plurality of locations visited by the user and may provide a semantic label to the visit history. The systems, devices, and methods may determine location related statistics for the visit history by analyzing the visit history and the semantic label. The systems, devices, and methods may generate one or more location-aware insights based on the location related statistics. The location-aware insights may identify patterns or location related statistics in the visit histories that may be related to the user's well-being or health.

### BACKGROUND

An increasingly fast-paced world and a collective sense of urgency has led to a recent rise of self-awareness methods and tools that provide users with insights into various aspects of their life. Knowing ourselves and reflecting upon our own behavior helps identify and filter out bad habits, as well as reevaluate our goals and refocus. Recent studies have associated our movement patterns and habits with our physical and mental well-being. The research has found that locations, either specific geolocations or their semantic type (e.g., recreational, retail, home, work, art location), may play a significant role in how individuals feel.

### BRIEF SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

One example implementation relates to a method for providing location-aware insights. The method may include determining a visit history for a user that includes a plurality of locations visited by the user over a time period by using location data received from a device of the user and determining the plurality of locations visited based on the location data. The method may include applying a plurality of semantic labels to the visit history, wherein each semantic label in the plurality of semantic labels corresponds to a location in the plurality of locations visited by the user. The method may include categorizing each location in the plurality of locations based on the plurality of semantic labels, wherein each location category has one or more corresponding environmental attributes. The method may include generating user routine data for the time period based on the visit history and the location categories. The method may include identifying a health or well-being deficiency based on the user routine data. The method may include generating an activity recommendation intended to assist the user in correcting the identified deficiency. The method may include presenting the activity recommendation to the user.

Another example implementation relates to a system. The system may include more processors; memory in electronic communication with the one or more processors; a visit detection model, a semantic enrichment component, an analytics component, and an insight component in electronic communication with the one or more processors and the memory; and instructions stored in the memory, the instructions executable by the one or more processors to cause one or more of the detection model, the semantic enrichment component, the analytics component, or the insight component to: determine a visit history for a user that includes a plurality of locations visited by the user over a time period by using location data received from a device of the user and determining the plurality of locations visited based on the location data; apply a plurality of semantic labels to the visit history, wherein each semantic label in the plurality of semantic labels corresponds to a location in the plurality of locations visited by the user; categorize each location in the plurality of locations based on the plurality of semantic labels, wherein each location category has one or more corresponding environmental attributes; generate user routine data for the time period based on the visit history and the location categories; generate an activity recommendation intended to assist the user in correcting the identified deficiency; and present the activity recommendations to the user.

Additional features and advantages will be set forth in the description which follows, and in part will be obvious from the description, or may be learned by the practice of the teachings herein. Features and advantages of the disclosure may be realized and obtained by means of the instruments and combinations particularly pointed out in the appended claims. Features of the present disclosure will become more fully apparent from the following description and appended claims or may be learned by the practice of the disclosure as set forth hereinafter.

### BRIEF DESCRIPTION OF THE DRAWINGS

In order to describe the manner in which the above-recited and other features of the disclosure can be obtained, a more particular description will be rendered by reference to specific implementations thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example implementations, the implementations will be described and explained with additional specificity and detail through the use of the accompanying drawings in which:

FIG. 1 illustrates an example environment for providing location-aware insights in accordance with implementations of the present disclosure.

FIG. 2 illustrates an example location graph for use with implementations of the present disclosure.

FIG. 3 illustrates an example graphical user interface screen of the insight dashboard in accordance with implementations of the present disclosure.

FIG. 4 illustrates an example graphical user interface screen of the insight dashboard in accordance with implementations of the present disclosure.

FIG. 5 illustrates an example graphical user interface screen with different location-aware insights presented on the insight dashboard in accordance with implementations of the present disclosure.

FIGS. 6A and 6B illustrate an example graphical user interface screen for use with a chatbot feature of the insight dashboard in accordance with implementations of the present disclosure.

FIGS. 7A-7G illustrate an example graphical user interface screen of the insight dashboard in accordance with implementations of the present disclosure.

FIG. 8 illustrates an example method for providing location-aware insights in accordance with implementations of the present disclosure.

### DETAILED DESCRIPTION

This disclosure generally relates to location-aware insights. Self-awareness is becoming a powerful tool for achieving a higher resilience to a growingly demanding world. Knowing ourselves and reflecting upon our own behavior helps identify and filter out bad habits, as well as reevaluate our goals and refocus. One way to achieve this is by self-tracking, that is, by tracking for instance our daily activities, sleep, and mood, among others. Wearable technology makes this kind of self-tracking and quantification easy. Beside physical activity, one of the most prominent signal that is often being tracked is location. Recent studies have associated our movement patterns and habits with our physical and mental well-being.

The research has found that locations, either specific geolocations or their semantic type (e.g., recreational, retail, home, work, art location), may play a significant role in how individuals feel. Moreover, depending on personalities and different situation (personal and workloads), everyone might need a different minimum amount of time spent at certain locations to achieve an optimal balance and wellbeing state (e.g., some individuals might need to spend more time outdoors than other individuals to get to a similar emotional and mental equilibrium state). In addition, the research has identified the importance of location-relevant environmental factors, such as, the air quality, the noise level, and the existence or access to green spaces in influencing a person's health and well-being. The research has also focused on associating geographic features and locations to mental health.

The present disclosure provides users with deeper insights about their visit patterns so that the users may retrospectively reflect upon their whereabouts and helps users better understand where and how the users spend their time. In addition, the present disclosure supports users in identifying visit patterns the users may want to change to improve a quality of life and/or promote a healthier lifestyle.

The present disclosure provides users with a deeper understanding of the users' visit and movement patterns by providing location-aware insights based on the visit patterns or movement histories of the users. The location-aware insights may identify patterns in visit histories that may be related to the user's well-being and/or health. The location-aware insights may provide recommendations or suggestions (e.g., activity recommendations) to the users to modify visit patterns of locations to improve the user's metal well-being or physical health. The location-aware insights may also be correlated with other aspects of the user's life to identify patterns that may be related to the user's well-being and/or health. For example, the location-aware insights are correlated with costs or expenses (e.g., transportation cost, food expenses, travel expenses) so that the users understand how visiting certain places may affects the user's money. In addition, the users may be provided with discounts or coupons for visiting certain places based on the location-aware insights.

The present disclosure provides an insight dashboard that highlights factors that have been proven to affect our well-being. The present disclosure utilizes an extended locations graph that goes beyond containing the typical hierarchical relations and considers additional semantic location attributes that are related with our well-being, such as, but not limited to, indoor spaces, outdoor spaces, green spaces, open spaces, and/or closed spaces. The present disclosure uses the semantic location attributes in analyzing the different locations visited by the user. The present disclosure focuses on well-being-related statistical features of the different locations, such as, but not limited to, location and visit frequency, duration, regularity, and/or periodicity. The insight dashboard presents the location-aware insights to the users in a variety of ways.

In accordance with the present disclosure, a personalized location-aware well-being insight dashboard may be generated for users so that the users may keep track of the quality time that the users invested during the day, week, month (in a retrospective manner) grouped by location category. The present disclosure may present location-aware insights on the insight dashboard based on a visit history of a user. An example location-aware insight includes notifying the user that “You have spent 25% of your time at retail locations and 68% of your time at home.” In addition to displaying the location-aware insights (e.g., 88% indoors, 12% outdoors), the insight dashboard may provide activity recommendations to the user to motivate the user to change visit patterns based on the suggestions. An example activity recommendations includes notifying the user that “You have spent 25% of your time at retail locations, 68% at home, but only 3% at parks. Why don't you take a stroll this weekend and get this 3% to 5%?”. As such, the insight dashboard may provide users with proactive recommendations for a healthier, location-aware way of living.

The present disclosure allows users to gain a better location-aware understanding by providing location-aware insights related to the user's mental well-being or physical health. The present disclosure allows users to better understand where and how the users spend their time and helps users understand whether any visit patterns need to change to promote a healthier lifestyle or improve the health or metal well-being of the users.

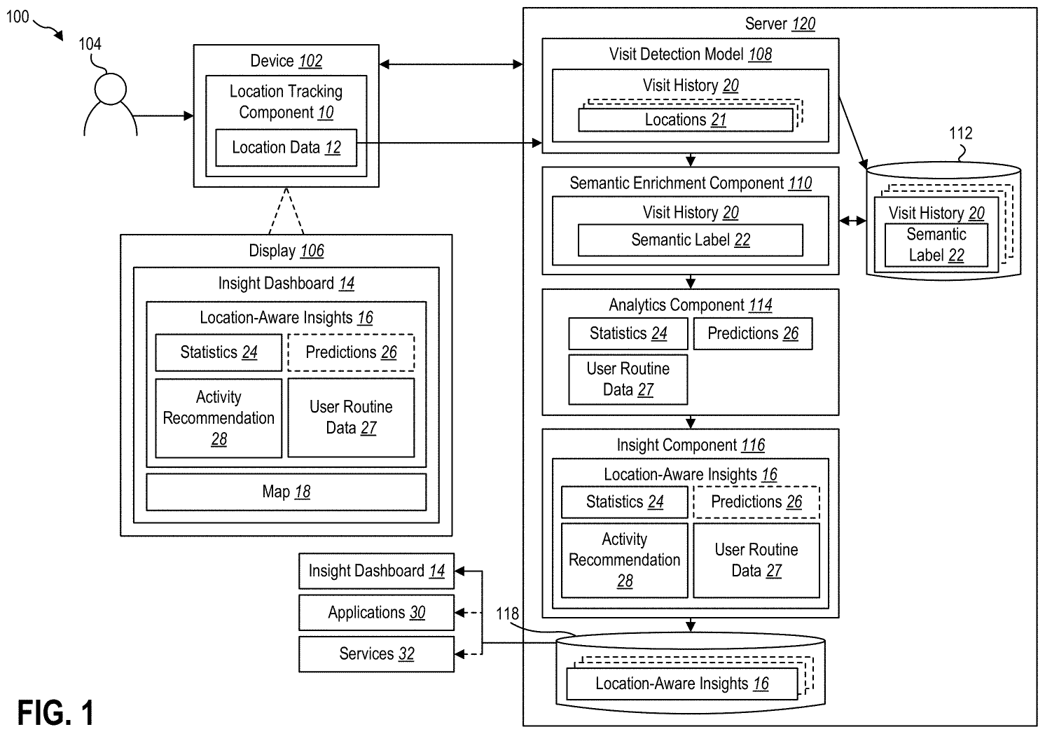

Referring now to FIG. 1, illustrated is an example environment 100 for providing location-aware insights 16. The environment 100 may have one or more devices 102 providing location data 12 of one or more users 104. The devices 102 may include a location tracking component 10 that tracks the location data 12 of the users 104. In some implementations, the location tracking component 10 is a global positioning system (GPS) locating tracking client. The location tracking component 10 collects the location data 12 and/or transportation mode data of the users 104 regularly. The intervals selected for collecting and sending the location data 12 activity registered by the device 102 may ensure that the data collection process is as accurate as possible and battery-efficient for the device 102. The device 102 may send the location data 12 to one or more servers 120 in the environment 100 for processing. The servers 120 may include a visit detection model 108 that receives the location data 12 and generates a visit history 20 for the location data 12. The visit detection model 108 may filter the location data 12 activity registered by the device 102 to identify and generate a corresponding set of visit histories 20 for the location data 12.

The visit detection model 108 may use spatial and temporal features of the location data 12 when generating the visit history 20. The visit history 20 may have an associated date and time (e.g., a start time and an end time) of the location data 12. In addition, the visit history 20 may include the coordinates of the arrival location. As such, the visit history 20 may identify a plurality of locations 21 that the user 104 visited based on the received location data 12.

The visit histories 20 for the users 104 may be stored in a datastore 112. The datastore 112 may include a visit history database that stores the visit histories 20 by each user 104. The visit histories 20 may be sorted by date and/or time. The datastore 112 may be an object storage, which may be accessed via an application programming interface (API) (e.g., a hypertext transfer protocol (HTTP) API) and/or a user-specific authentication token.

The visit histories 20 for the users 104 may be accessed by a semantic enrichment component 110. The semantic enrichment component 110 may retrieve the visit histories 20 from the datastore 112. The semantic enrichment component 110 may also receive the visit histories 20 from the visit detection model 108.

The semantic enrichment component 110 may classify the locations 21 of each visit history 20 and may enrich each visit history 20 with a corresponding semantic label 22. The semantic label 22 may include a name of a business or place if the location 21 is for a public location. For example, the semantic enrichment component 110 calls a location recognition API to retrieve the name of the business or place. The semantic label 22 may also include a user-defined custom label, such as, but not limited to, home, work, or school for the location 21. The user-defined custom labels may be automatically inferred by a rule-based or machine learning-based algorithm. For example, the algorithm names a location 21 where the users 104 consistently spend their nights “home” and a location 21 where the users 104 spend between 8 am and 5 pm during the week “work.” In addition, the users 104 may provide the custom labels for personal places (e.g., Mom's home, Mary's home) using, for example, the insight dashboard 14. The users 104 may also provide a custom label for a place that has a business name, and the system may replace the business name with the user-defined custom label (e.g., the user may provide the custom label “My Coffee Shop” and the system may replace the business name of the coffee shop with the custom label).

The semantic label 22 may also include a location category (e.g., park, restaurant, office, etc.). The semantic enrichment component 110 may use location graphs to produce the corresponding location categories for the different locations 21 included in the visit histories 20. As such, the semantic label 22 may assign each location 21 included in the visit history 20 a location name and a location type or category.

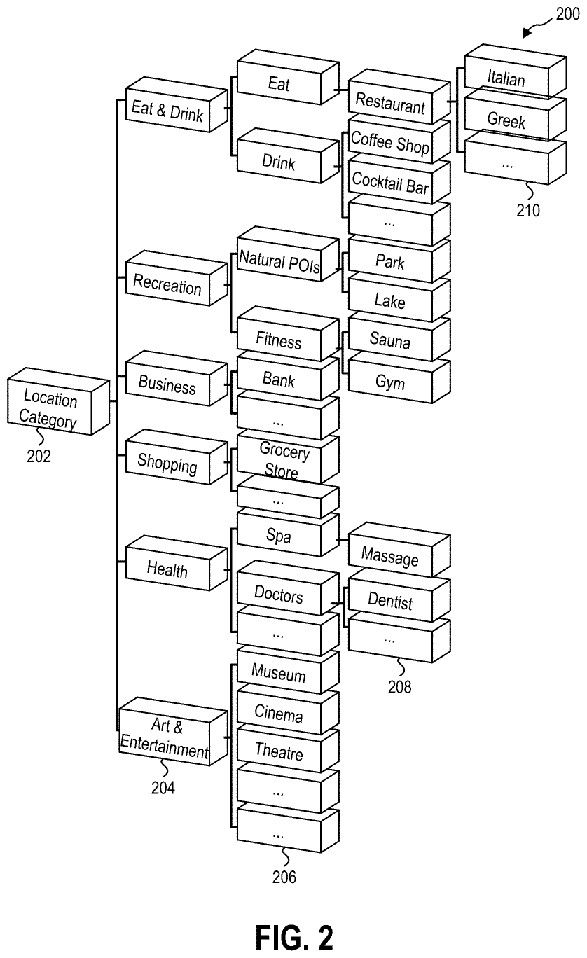

Referring now to FIG. 2, illustrated is an example location graph 200 for use by the semantic enrichment component 110. The location graph 200 may illustrate the taxonomic relations of the location categories and provide additional information about the different locations. The location graph may divide the location category 202 into subcategories 204 (e.g., Eat and Drink, Recreation, Business, Shopping, Health, and Art and Entertainment). The subcategories 204 may be further divided into other subcategories 208 (e.g., Restaurants, Coffee Shop, Cocktail Bars, Parks, Lakes, Sauna, Gym, Massage, and Dentist). In addition, the subcategories 208 may be further subdivided into other subcategories 210 (e.g., Italian, Greek). As such, the location graph 200 may provide the hierarchy relations of the location category 202. In addition, the location graph 200 may provide additional semantic relations to other categories. The location graph 200 may provide additional details for the location category 202, such as, but not limited to, a type of food or drink, adjectives describing the location category, and/or location attributes.

Referring to FIG. 1, the semantic enrichment component 110 may use one or more location graphs 200 for generating the semantic label 22 for the visit histories 20. In addition, the semantic enrichment component 110 may use one or more knowledge graphs of other domains that that may store and represent data of relevant domains, such as transportation. An example knowledge graphs includes a graph representing a taxonomy of different transportation modes ((1) public->bus, train->metro, subway; (2) private->4 wheel->car, truck and 2 wheel->motorcycle, bike, and boat versus ferry, etc.) The knowledge graphs may also contain additional attributes for each transportation mode, such as, fast, slow, expensive, good for the environment, bad for the environment, good for health (bike, kayak), etc. The semantic enrichment component 110 may use the location graphs and/or the knowledge graphs to identify location attributes for the locations 21 of the visit history 20. The semantic label 22 may include location attributes with adjectives or details describing the locations of the visit history 20. The location attributes may include ambient factors or environmental attributes, such as, but not limited to, air quality, light conditions, noise, indoor spaces, outdoor spaces, open spaces, closed spaces, green spaces, grey spaces, popular spaces, less popular spaces, bright spaces, dark spaces, new spaces, and/or old spaces. In addition, the location attributes may include cost relevant attributes for the locations 21 of the visit history 20.

The semantic enrichment component 110 may enrich the visit histories 20 with a semantic label 22 that provides more comprehensive information for the locations 21 included in the visit histories 20. The semantic enrichment component 110 may add the semantic labels 22 to the visit histories 20 stored in the datastore 112. In addition, the semantic enrichment component 110 may send the visit histories 20 with the semantic labels 22 to an analytics component 114. The analytics component 114 may also access the visit histories 20 and the semantic labels 22 from the datastore 112.

The analytics component 114 may analyze the visit histories 20 and the semantic labels 22 to identify user routine data 27 that may impact the user's 104 well-being or emotional health. The user routine data 27 may include visit patterns of the user 104 to different locations 21 of the visit history 20 over a time period. The analytics component 114 may determine location related statistics 24 about the visit histories 20 using the semantic labels 22. The analytics component 114 may use the location related statistics 24 to determine the user routine data 27. The analysis performed by the analytics component 114 highlights health and/or mood-relevant patterns in the user routine data 27 of the users 104. In addition, the analytics component 114 may use the statistics 24 to infer the level of well-being of the users 104 (e.g., the physical health, and/or emotional health of the user 104). The analysis of the user routine data 27 (e.g., the visit patterns) may also be used by the analytics component 114 to infer a mood of the users 104 and/or to indicate certain personality traits of the users 104 (e.g., extroversion). The analysis may be used to choose a correct timing to interrupt the user 104 (e.g., to inform the user 104 about an event that might be interesting, or when it might be a good time to take a break and where to take the break).

The statistics 24 may identify a frequency of the visits to a certain location type and/or when the visits occur (e.g., which day and/or time of day). The statistics 24 may also identify a duration of the visits (e.g., how long are the visits and when do the visits peak). The duration may also indicate a transit time for the visit (e.g., how long does it take to get to the location). The statistics 24 may also identify a regularity of the visit patterns (e.g., whether the visit patterns regularly occur or are irregular). The statistics 24 may also identify a variety of the visit patterns (e.g., an amount of visit locations during a time range and a ratio of new and old places visited during the time range). The statistics 24 may also identify location attributes of the visit patterns (e.g., green space, grey space, noisy, quiet, open space, closed space). The statistics 24 may also identify sequencing and association mining of the visit patterns (e.g., 90% of the time when going to the movies a user visits a restaurant after the movie). Other factors of the user routine data 27 and the semantic labels 22 may be identified by the analytics component 114 that may impact the well-being or emotional health of the user 104 and may be included in the statistics 24.

The analytics component 114 may also analyze the user routine data 27 and the semantic labels 22 to generate any predictions 26 about the user 104. The analytics component 114 may estimate the user's 104 future visit patterns based on the analysis of the user routine data 27 and the semantic labels 22. The predictions 26 may provide the users 104 with a possibility to adapt in advance potential negative visit patterns (e.g., to add a visit to a park into the day based on a low prediction 26 that the user will not go outside today).

The analytics component 114 may operate at multiple semantic levels by covering the individual location instances included in the visit histories 20 and processing the location instances semantic types to provide more insightful information regarding the users' 104 movement and visit patterns in the visit histories 20. In an implementation, the analytics component 114 may use a set of Markov models (a Markov Chain (MCM), a hidden MC (HMC), and a Mixed MC (MMC)) to perform the processing of the visit histories 20 and the semantic labels 22.

An example use case includes the analytics component 114 outputting a statistic 24 for how many times the user 104 visited a certain coffee shop since last month and outputting a statistic 24 with the overall times the user 104 has visited any coffee shop, or from a broader point of view, outputting a statistic 24 with the overall time the user 104 has visited any Eat and Drink location type (e.g., bars, night clubs and food locations) as well. The statistics 24 may also contain other attributes about the coffee shops and/or the eating establishments that the user 104 visited (e.g., open, green, quite if the coffee shop is located out of town in the open countryside).

The analytics component 114 may send the user routine data 27, the statistics 24 and/or the predictions 26 to an insight component 116. The insight component 116 may aggregate the user routine data 27, the statistics 24 and/or the predictions 26 for the visit histories 20 of the user 104 and may generate one or more location-aware insights 16 based on the statistics 24 and/or the predictions 26. The insight component 116 may identify the user routine data 27, the statistics 24 and/or the predictions 26 correlated to a health or mental well-being of the user 104 in generating the location-aware insights 16. The location-aware insights 16 may highlight the user routine data 27 (e.g., visit patterns), the statistics 24, and/or the predictions 26 of the user 104 to different locations 21 that may affect the mental well-being or health of the user 104. The location-aware insights 16 may highlight or summarize the user routine data 27 of the user 104 in a relatable manner so that the user 104 may easily identify different statistics 24 and/or predictions 26 related to the different locations 21 and/or location types of the visit histories 20.

For example, the location-aware insights 16 highlight an amount of time the user 104 spent in outdoor spaces compared to the amount of time the user 104 spent in indoors spaces. The location-aware insights 16 may highlight an amount of time the user 104 spent shopping this week compared to the amount of time the user 104 spent shopping the previous week. The location-aware insights 16 may identify that the user 104 has visited the same locations for three weeks straight without any variation in locations.

The location-aware insights 16 may identify how regular (or rare) are the user's 104 visits to certain locations 21 or location types (e.g., gym, parks, restaurants, entertainment venues, hotels, work, etc.). The location-aware insights 16 may also identify when the user's 104 visits at certain places peak with respect to time of day and a day of the week. The time of day and day of the week for the visits may be relevant to traffic-related stress and/or costs associated with the visit (e.g., transportation costs and/or an amount of money spent at the location). The location-aware insights 16 may also identify a duration of the visit (e.g., how long is the visit to a specific location or a location type). The location-aware insights 16 may also identify how much time the user 104 lost in transit during the week for the different visits. The location-aware insights 16 may also identify an amount of time the user spends in public places and private places (residential locations).

The location-aware insights 16 may also identify the last time the user 104 was out of town. The location-aware insights 16 may also identify how much time the user 104 spent in green spaces for an interval of time (e.g., previous week, previous month, previous two weeks). The location-aware insights 16 may also identify a variety of visit locations for the user 104 (e.g., a ratio of the user's 104 time with respect to indoor spaces versus outdoor spaces, open spaces versus closed spaces, and/or new locations versus the same locations). As such, the location-aware insights 16 may present different well-being statistics 24 and/or other factors related to the visit histories 20 of the user 104 that identify how the user 104 is spending time and in what type of places the user 104 is spending time.

The location-aware insights 16 may also include one or more activity recommendations 28 to improve the well-being or health of the user 104. The activity recommendations 28 may provide proactive recommendations for a healthier, location-aware way of living. Example activity recommendations 28 include, booking in advance a free day in the park, changing a visit pattern, trying a new activity, and/or visiting a new or different location. The activity recommendations 28 may be tailored to the user 104. For example, the activity recommendations 28 recommend that the user 104 become more social or adventurous and provide recommendations for specific location based activities or location types for the activity recommendations 28.

The insight component 116 may also aggregate the user routine data 27, the statistics 24 and/or the predictions 26 for the visit histories 20 of a plurality of users 104 and may generate one or more shared location-aware insights 16 based on the user routine data 27, the statistics 24 and/or the predictions 26 for the plurality of users 104.

The insight component 116 may output the location-aware insights 16 on an insight dashboard 14. The insight dashboard 14 may be a web or native application dashboard. The insight dashboard 14 may present the location-aware insights 16 in an easy to understand manner so that the user 104 may easily identify or understand the user routine data 27, the statistics 24, the activity recommendations 28, and/or other factors of the visit histories 20 that may affect the user's 104 mental well-being or health.

The insight dashboard 14 may use a variety of visuals or modalities for presenting the location-aware insights 16. Examples include graphics, animations, charts, text, speech, reports, and/or push notifications. The insight dashboard 14 may represent different types of information for the location-aware insights 16 using different modalities. For example, statistics 24 may be presented using graphics or charts and interesting facts may be presented using text. One example includes using a pie chart or doughnut chart for the frequency and duration statistics 24. Another example includes using a stacked line chart or bar chart for the peaks statistics 24 (e.g., by time of day or day of the week). Another example includes using a spider chart or radar chart for the location attributes information included in the statistics 24. Another example includes using a calendar chart, a timeline chart, or time machine chart for tracking the visit patterns over a time interval. Another example includes using a map to display the different locations visited and provide spatial awareness of the different locations. Another example includes using a gauge or progress chart for any goals (e.g., health goals, location type goals) the users 104 have set. Another example includes using a word cloud with the different location types for the different visits. As such, the insight dashboard 14 may use diverse representations to present the location-aware insights 16 to the user 104.

The insight dashboard 14 allows user interactivity and supports gestures by the users 104 (e.g., pinching zooming, touching, dragging, scrolling, and/or swiping). A date range picker provided by the insight dashboard 14 allows the users 104 to select either individual dates or a date range for generating the location-aware insights 16. The date range may be a set of predefined time intervals (e.g., one week, one month, three months, six months, one year) to help the users 104 easily identify the location-aware insights 16 that may affect the health or well-being of the user 104 during the time intervals. A time axis slider may allow the users 104 to slide back and forth in time (e.g., move back a month or move forward a week). As the user slides back and forth in time, a map 18 may update with different locations that the user visited during the selected time interval. As such, the time axis slider may allow the users 104 to easily track movement patterns and see changes in the location visits over a time interval by moving backwards or forwards in time.

In addition, the user 104 may view the locations visited on a map and may filter the locations visited by date and/or location type. For example, the user 104 filters their locations to display all park visits in the last 2 weeks. A popup window for each displayed location on the map may display information relevant to the visit(s) that took place at this specific location (e.g., location name(s), visit date(s), duration(s), start time(s), end time(s), popularity, location attributes, and/or other statistics 24 for the visit).

The insight dashboard 14 may also display timeline charts that allows the user 104 to compare visit patterns across different time segments. The user 104 may use the timeline charts to identify peak and/or irregular behaviors across different time segments. For example, the user 104 scrolls the timeline backwards and/or forwards in to track the visit pattern flows of the different locations visited by the user 104.

The insight dashboard 14 may also provide rewards and/or incentives to the users 104 for following the activity recommendations 28 or recommendations from the location-aware insights 16. The insight dashboard 14 may also provide rewards and/or incentives to the users 104 for achieving goals set for visits. For example, the user 104 has a health goal and the insight dashboard 14 provides rewards or incentives to the user 104 for visiting locations to achieve the health goal (e.g., visiting parks or gyms for a health goal of being more active, visiting doctor offices, visiting certain restaurants, etc.).

The insight dashboard 14 may be customized to preferences of the user 104. The user 104 may select different charts for viewing the same location-aware insights 16. In addition, the dashboard elements may be rearranged based on the preferences of the user 104. In addition, the insight dashboard 14 may be adapted to the display 106 of a device 102. For example, the screen size increases or decreases based on the device 102 associated with the display 106 (e.g., phone, tablet, desktop). Another example includes the orientation of the insight dashboard 14 changes based on an orientation of the device 102 associated with the display 106.

The insight dashboard 14 may also provide shared location-aware insights 16 for a group of users 104 (e.g., family, friends, or contacts). For example, parents receive location-aware insights about their children (e.g., an amount of time the children spent outdoors, at school, at entertainment venues, at friend's houses). A family may have a shared insight dashboard 14 that tracks common movement and/or visit patterns of the entire family. The location-aware insights 16 may identify time spent together as a family at certain locations (e.g., home, parks, shopping, restaurants). The location-aware insights 16 may be used to set goals (e.g., spend more time outside) for the family and the insight dashboard 14 may track the progress towards the goals for each of the family members. The insight dashboard 14 may also be used to create competitions between the family members for achieving the goals and/or providing rewards for achieving the goals. In addition, the insight dashboard 14 may provide the shared location-aware insights 16 in an abstracted manner to protect the privacy of other users (e.g., provide the shared location-aware insights 16 in a general manner without identifying specific location names).

The insight component 116 may also store the location-aware insights 16 in one or more datastores 118. The location-aware insights 16 may be stored by each individual user 104 in the datastore 118. The location-aware insights 16 may be accessed from the datastore 118 by the insight dashboard 14 and/or other applications 30 or services 32.

The location-aware insights 16 may also be used by other applications 30 and/or services 32. The applications 30 and/or services 32 may aggregate the location-aware insights 16 from a plurality of users 104 to provide discounts or offers to promote an activity or a business based on the information provided in the location-aware insights 16. For example, the applications 30 and/or services 32 provide coupons or incentives for a region of users 104 based on the location-aware insights 16 for the region (e.g., a local business or local outdoor activity). The promotions or discounts may also be tailored to specific to the user 104 based on the location-aware insights 16 for the user 104.

Another example includes other applications 30 providing calendar updates and/or notices for the activity recommendations 28 (e.g., a calendar application on the device 102 of the user 104 schedules time to take a walk in the middle of the day around lunchtime or provides a notice that the user 104 has a break in the schedule and it might be nice to get outside). Another example includes the user 104 setting goals (e.g., health goals) and the applications 30 or services 32 helping the user 104 track progress for the goals based on the location-aware insights 16.

The insight dashboard 14 may integrate with the other applications 30 or services 32 to provide additional information with the location-aware insights 16. For example, the insight dashboard 14 coordinates with a map application to display maps 18 related to the location-aware insights 16 (e.g., showing on the maps 18 the locations identified in the location-aware insights 16). Another example includes the insight dashboard 14 presenting the coupon or offers nearby the locations on the maps 18 for the different location-aware insights 16.

The insight dashboard 14 may integrate with other applications 30 or services to provide information to the users 104 correlating expenses for the different location-aware insights 16. For example, the insight dashboard 14 provides the amount of money that the user 104 spent at coffee shops last week ($40) and the amount of money that the user 104 spent at coffee shops this week ($50). Another example includes the insight dashboard 14 providing the transportation costs that the user 104 spent during the week for the different locations visited by the user 104.

The environment 100 may have multiple machine learning models running simultaneously. One or more of the visit detection model 108, the semantic enrichment component 110, the analytics component 114, and/or the insight component 116 may have one or more machine learning models that run concurrently to perform the processing. In addition, the environment 100 may implement a federated learning approach. A federated learning approach may be used so that the location data 12 does not leave the user's 104 device 102 to be trained and/or inferred by the various models and/or components of the environment 100. For example, the federated learning approach is used when generating multi-user insights. In some implementations, one or more computing devices (e.g., servers 120 and/or devices 102) are used to perform the processing of environment 100. The one or more computing devices may include, but are not limited to, server devices, personal computers, a mobile device, such as, a mobile telephone, a smartphone, a PDA, a tablet, or a laptop, and/or a non-mobile device. The features and functionalities discussed herein in connection with the various systems may be implemented on one computing device or across multiple computing devices. For example, the visit detection model 108, the semantic enrichment component 110, the analytics component 114, the insight component 116, and/or the datastores 112, 118 are implemented wholly on the same computing device. In an implementation, the visit detection model 108, the semantic enrichment component 110, the analytics component 114, the insight component 116, and/or the datastores 112, 118 are implemented on the device 102 and the processing of the environment 100 takes place locally on the device 102. In another implementation, the visit detection model 108, the semantic enrichment component 110, the analytics component 114, the insight component 116, and/or the datastores 112, 118 are implemented on the same server 120. Another example includes one or more subcomponents of the visit detection model 108, the semantic enrichment component 110, the analytics component 114, the insight component 116, and/or the datastores 112, 118 implemented across multiple computing devices (e.g., across multiple servers 120). Moreover, in some implementations, the visit detection model 108, the semantic enrichment component 110, the analytics component 114, the insight component 116, and/or the datastores 112, 118 are implemented or processed on different server devices of the same or different cloud computing networks.

In some implementations, each of the components of the environment 100 is in communication with each other using any suitable communication technologies. In addition, while the components of the environment 100 are shown to be separate, any of the components or subcomponents may be combined into fewer components, such as into a single component, or divided into more components as may serve a particular embodiment. In some implementations, the components of the environment 100 include hardware, software, or both. For example, the components of the environment 100 may include one or more instructions stored on a computer-readable storage medium and executable by processors of one or more computing devices. When executed by the one or more processors, the computer-executable instructions of one or more computing devices can perform one or more methods described herein. In some implementations, the components of the environment 100 include hardware, such as a special purpose processing device to perform a certain function or group of functions. In some implementations, the components of the environment 100 include a combination of computer-executable instructions and hardware.

As such, the environment 100 may be used to highlight location-relevant facts to the users 104 using location-aware insights 16 to help the users 104 easily understand how the users 104 distribute their time at different locations 21 and how their visits to different locations 21 may have an impact on their well-being or health. The environment 100 may help the users 104 reflect on their visit and movement behavior to help the users 104 find balance and a well-being state. For example, the user 104 alters a visit pattern to reduce an amount of time in traffic.

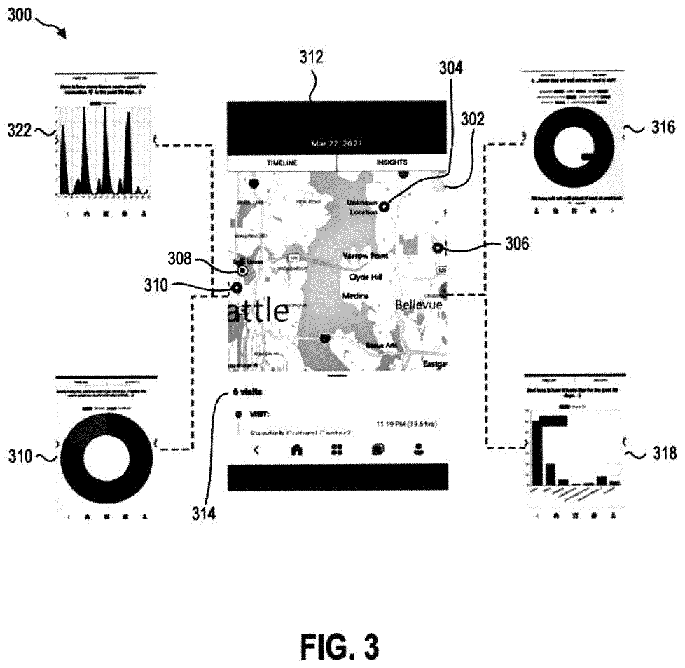

Referring now to FIG. 3, illustrated is an example graphical user interface (GUI) screen 300 of the insight dashboard 14 (FIG. 1). The GUI screen 300 may be presented on a display 106 of a device 102 (FIG. 1) of the user 104 (FIG. 1). The GUI screen 300 includes a map 302 displaying different visit locations (304, 306, 308, 310) that the user 104 visited for a selected date 312 (e.g., Mar. 22, 2021). Below the map 302, the GUI screen 300 may provide a list 314 identifying the number of visit locations (304, 306, 308, 310) and the name of the different visit locations (304, 306, 308, 310). The list 314 may also provide statistics 24 or other information for the different visit locations (304, 306, 308, 310). For example, the list 314 provides the visit time for each location and the duration of the visit. The list 314 may also provide the address of each location.

The user 104 may be able to select a different date or move the selected date 312 forwards and/or backwards in time. As the user 104 changes the selected date 312, the map 302 may be automatically updated with the different visit locations (304, 306, 308, 310) for the different date. Thus, the user 104 may see the changes in the locations on the map 302 over a time interval.

The GUI screen 300 may also present different charts and/or graphs presenting statistics 24 for the different visit locations (304, 306, 308, 310) displayed on the map 302. The chart 316 may use a doughnut chart to present statistics 24 for the different visit locations (304, 306, 308, 310), such as, an amount of time the user 104 spent at home, the office, shopping, arts and entertainment, recreation based on the different visit locations (304, 306, 308, 310) for the selected date 312. The chart 318 may use a bar chart to illustrate the same statistics 24 shown in the chart 316 for the different visit locations (304, 306, 308, 310). As such, the GUI 300 may use different visuals to convey the same statistics 24 for the different visit locations (304, 306, 308, 310). For example, the chart 316, 318 selected for presentation on the insight dashboard 14 is selected based on the preferences of the user 104.

The chart 320 may use a doughnut chart to illustrate statistics 24 for the different visit locations (304, 306, 308, 310) relating to an amount of time spent outdoors versus an amount of time spent indoors. The graph 322 illustrates statistics 24 for the different visit locations (304, 306, 308, 310) relating to an amount of time spent on recreation activities. The different charts 316, 318, 320 and/or graphs 322 may be presented when the user selects different icons on the insight dashboard 14. The user 104 may also select different time intervals for the statistics 24 presented on the charts 316, 318, 320 and/or graphs 322. The charts 316, 318, 320 and/or graphs 322 may be presented in an overlay on the map 302 nearby or adjacent to the different visit locations (304, 306, 308, 310). In addition, the charts 316, 318, 320 and/or graphs 322 may be presented instead of the map 302 on the insight dashboard 14.

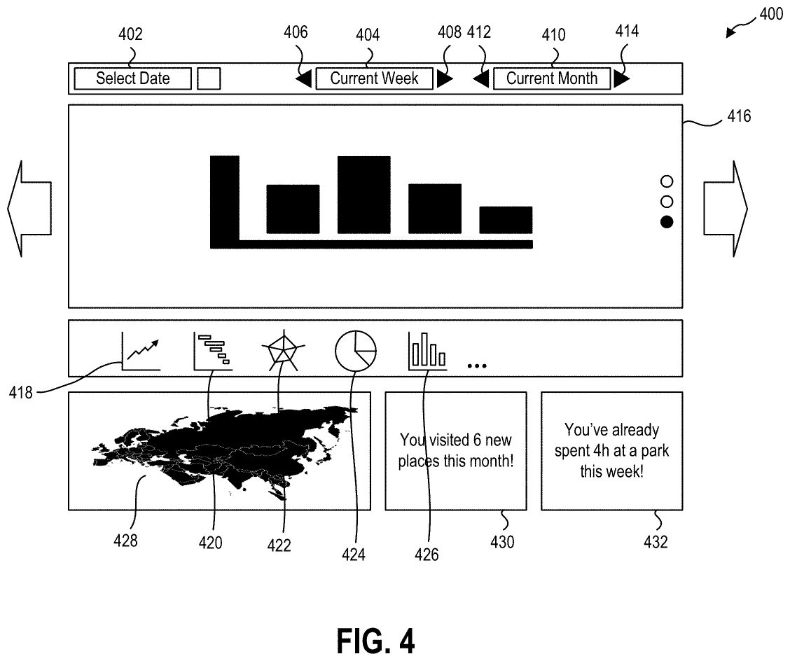

Referring now to FIG. 4, illustrated is an example graphical user interface (GUI) screen 400 of the insight dashboard 14 (FIG. 1). The GUI screen 400 may be presented on a display 106 of a user's 104 device 102 (FIG. 1). The GUI screen 400 may present one or more location-aware insights 16 (FIG. 1) on the insight dashboard 14.

The GUI screen 400 may include a date input field 402 where the user 104 may select a date or time interval for the location-aware insights 16. The date input field 402 may use predefined time interval ranges (e.g., a week, a month, two weeks, three months, six months, a year). The date input field 402 may present the predefined time interval ranges (e.g., in a list) and the user 104 may select one of the predefined time interval ranges provided in the date input field 402. In addition, the user 104 may enter in the date or the time interval in the date input filed 402.

The GUI screen 400 may present the location-aware insights 16 for the selected date or time interval. The GUI screen 400 may have a visual carousel 416 that presents the different location-aware insights 16. For example, a bar graph is presented in the visual carousel 416 with the statistics 24 (FIG. 1) and/or other information related to the location-aware insights 16 for the selected date or time interval.

The visual carousel 416 may be a nested carousel that allows the user 104 to swipe horizontally to switch between different chart or graph types. In addition, the user 104 may swipe vertically to switch between different domains for the same chart or graph (e.g., frequency-duration, time of day, or day of week).

Thumbnails 418, 420, 422, 424, 426 may be displayed below the visual carousel 416 identifying the different charts or graphs available for the location-aware insights 16. In addition, the thumbnails 418, 420, 422, 424, 426 may present an order that the different charts or graphs may be displayed. For example, if the user 104 swipes horizontally, the graph associated with the thumbnail 418 (e.g., the line graph) is displayed next. If the user 104 swipes horizontally again, the graph associated with thumbnail 420 (e.g., the horizontal bar graph) is displayed next. The user 104 may also select different thumbnails 418, 420, 422, 424, 426 to have a different chart type or graph type displayed in the visual carousel 416. For example, if the user selects thumbnail 424, a pie chart is displayed in the visual carousel 416 for the location-aware insights 16.

The thumbnails 418, 420, 422, 424, 426 included on the insight dashboard 14 may be selected based on the preferences of the user 104. The user 104 may add additional thumbnails or remove thumbnails from the insight dashboard 14. In addition, the order of the thumbnails 418, 420, 422, 424, 426 may be based on the preferences of the user 104. The user 104 may rearrange the order of the thumbnails 418, 420, 422, 424, 426 presented on the insight dashboard 14.

The GUI screen 400 may also display a map 428 displaying the visit locations for the selected date range or the selected chart displayed on the visual carousel 416. The map 428 may automatically update the different visit locations as the user changes the selected date or time interval and/or changes the selected chart for presentation on the visual carousel 416.

The GUI screen 400 may present the current week 404 and/or the current month 410 for the selected date or selected time interval. The user may use input icons (e.g., arrows 406, 408, 412, 414) to change the selected date or selected time. For example, the user 104 uses arrows 406, 408 to change the current week 404 forwards or backwards in time, and the user 104 uses arrows 412, 414 to move the current month 410 forwards or backwards in time. The map 428 may be interactive and the visit locations displayed on the map 428 may update based on the changes made by the user 104. For example, the user 104 selects last week as the time interval and the map 428 displays all the locations that the user 104 visited last week. The user 104 may use the arrow 408 to change the time interval to this week and some of the locations that the user 104 visited last week (that the user 104 did not visit this week) may be removed or disappear from the map 428 when the user changes the time interval. In addition, new locations that the user 104 visited this week (that this user 104 did not visit last week) may appear on the map 428 when the user changes the time interval. As such, the GUI screen 400 provides the user 104 with an interactive timeline tracking different visit patterns as the user scrolls or changes the timeframes associated with the location-aware insights 16 presented on the insight dashboard 14.

The GUI screen 400 may also include interesting facts 430, 432 and/or any outlier behaviors associated with the location-aware insights 16. For example, the interesting fact 430 identifies that the user 104 visited six new places this month, and the interesting fact 432 may identify that the user 104 has spent four hours at a park this week. The GUI screen 400 may also present metadata providing information about the actual visit of the user 104. The information may include, but is not limited to, start time, end time, duration, location name, location semantics, a popularity score of the location, and/or location attributes.

The user 104 may rearrange how the information is presented on the GUI screen 400 based on the preferences of the user 104. For example, the user 104 moves the map 428 above the visual carousel 416. In addition, the user 104 may switch the placement of the interesting facts 430, 432 and/or outlier behavior with the placement of the map 428. As such, the insight dashboard 14 may be customized or tailored for different users' preferences.

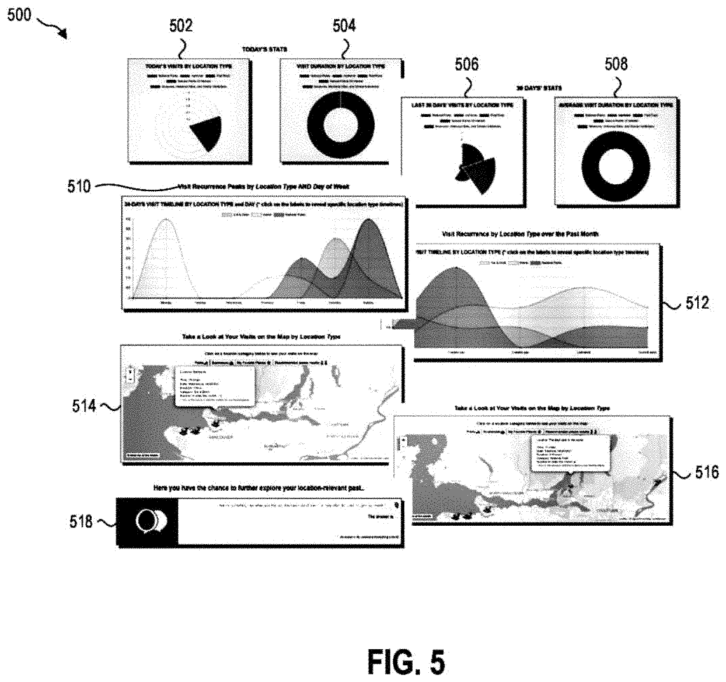

Referring now to FIG. 5, illustrated is a graphical user interface screen 500 with different location-aware insights 16 (FIG. 1) presented using a variety of visuals on the insight dashboard 14 (FIG. 1). The insight dashboard 14 may be presented on a display 106 of a device 102 (FIG. 1) of the user 104 (FIG. 1). The location-aware insights 16 may be presented to the user 104 on the insight dashboard 14 using a variety of different visuals.

Chart 502 uses a radar chart to illustrate the statistics 24 for the location-aware insights 16 for today's visits by location type (e.g., National Parks, Home, Fast Food, Natural Points of Interest, Museums). The radar chart may also illustrate location attributes (e.g., public, private, business, wellness, noisy, quite, etc.). Chart 504 uses a doughnut chart to illustrate the statistics 24 for the location-aware insights 16 for today's visits by location type. The charts 502, 504 may show the same statistics 24 for the location-aware insights 16 using different visual representations.

Chart 506 uses a radar chart to illustrate the statistics 24 for the location-aware insights 16 by location type (e.g., National Parks, Home, Fast Food, Natural Points of Interest, Museums) for the past thirty days. Chart 508 uses a doughnut chart to illustrate the statistics 24 for the location-aware insights 16 by location type (e.g., National Parks, Home, Fast Food, Natural Points of Interest, Museums) for the past thirty days. As such, the charts 506, 508 present the same statistics 24 for the location-aware insights 16 using different visual representations. Moreover, by comparing the charts 502, 504 with the charts 506, 508, the user 104 may easily identify the difference in the statistics 24 for the same location types over thirty days.

Graph 510 illustrates the statistics 24 for the location-aware insights 16 for visit recurrence peaks by location type (e.g., Eat and Drink, Home, National Parks) and day of the week for a thirty day time interval. Graph 512 illustrates the statistics 24 for the location-aware insights 16 for visit recurrence peaks by location type (e.g., Eat and Drink, Home, National Parks) over the past month. As such, the graphs 510 and 512 may show different statistics 24 for the location-aware insights 16 for the same time interval.

The statistics 24 for the location-aware insights 16 may also be presented on the maps 514, 516. The user 104 may look at their visits on the maps 514, 516 by location type (e.g., parks, businesses, favorite places). The user 104 may select a location type and view the locations for the selected location type on the map 514, 516. Different locations may be shown on the maps 514, 516 for different location types. Overlays may be presented nearby or adjacent to the locations on the maps 514, 516. The overlays may include statistics 24 and/or other information for the visits (e.g., time the visit occurred, the date of the visit, a duration of the visit, a category of the location, the number of visits this month to the location).

A chatbot 518 or other interface may be used to present the location-aware insights 16. The user 104 may ask questions to the chatbot 518 and may receive answers to the questions based on the location-aware insights 16. The user 104 may type questions into the chatbot 518 and the answers may be presented using text on the GUI screen 500. In addition, the user 104 may use speech to ask the chatbot 518 questions about the user's visit histories. Audio inputs on the device 102 may capture the question and text-to-speech processing may convert the question into text. The answers may be provided to the user 104 by audio or may be presented with text on the GUI screen 500.

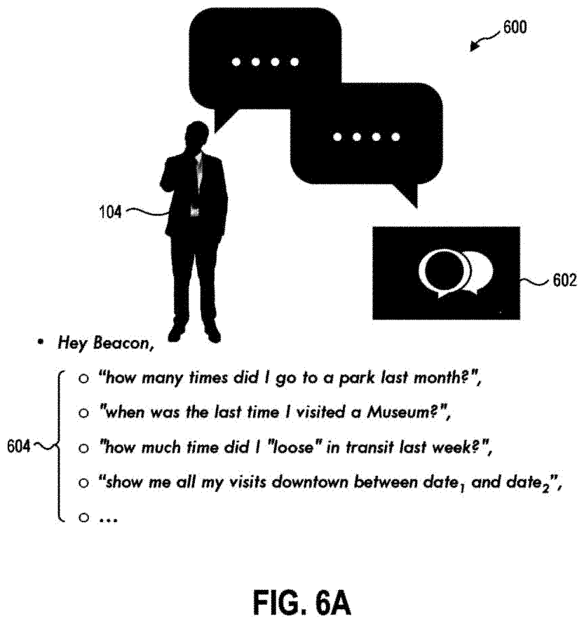

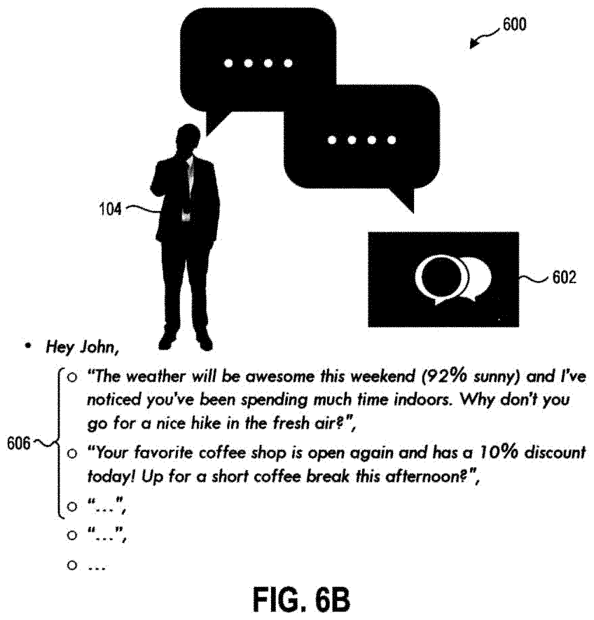

Referring now to FIGS. 6A and 6B, illustrated is an example graphical user interface (GUI) screen 600 of a chatbot for use with the insight dashboard 14 (FIG. 1). The GUI screen 600 may be presented on a display 106 of the user's device 102 (FIG. 1). The insight dashboard 14 may have a chatbot 602 that the user 104 may ask various questions 604 about the user's visit histories 20 (FIG. 1) and receive a response from the chatbot 602. An example question 604 includes “How many times did I go to the park last month?”. Another example question 604 includes “When was the last time I visited a museum?”. Another example question 604 includes “How much time did I lose in transit last week?”. Another example question 604 includes “Can you show me all my visits downtown between a first date and a second date?”.

The insight dashboard 14 may retrieve one or more location-aware insights 16 for the questions 604 and may display the location-aware insights 16 on a graphical user interface (e.g., GUI 300, GUI 400, GUI 500) in response to the questions 604. In addition, the insight dashboard 14 may provide the location-aware insights 16 to the chatbot 602 to respond to the user 104 (e.g., via text or via audio).

In addition, the chatbot 602 may provide one or more activity recommendations or suggestions 606 related to one or more location-aware insights 16 to the user 104. An example recommendation or suggestion 606 includes “The weather will be awesome this weekend (92% sunny) and I've noticed that you have been spending a lot of time indoors. Why don't you go for a nice hike in the fresh air?”. Another example recommendation or suggestion 606 includes “Your favorite coffee shop is open again and has a 10% discount today! Are you up for a short coffee break this afternoon?”.

The insight dashboard 14 may retrieve the activity recommendations or suggestions 606 based on the location-aware insights 16 for the user 104 and may provide the activity recommendations or suggestions 606 to the chatbot 602. The chatbot 602 may provide the activity recommendations or suggestions 606 to the user 104 via audio or text displayed on the GUI 600.

As such, the chatbot 602 feature of the insight dashboard 14 may provide another way for the user 104 to receive the location-aware insights 16 and interact with the insight dashboard 14.

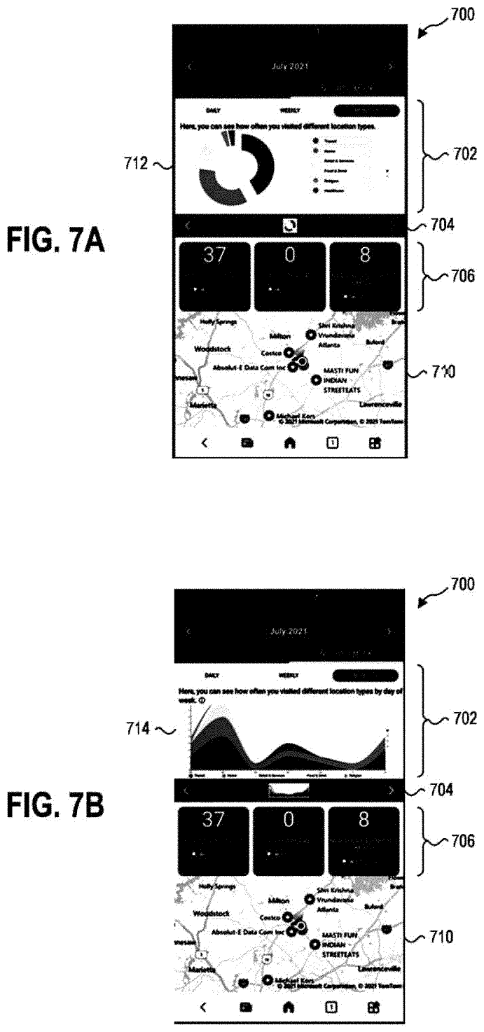

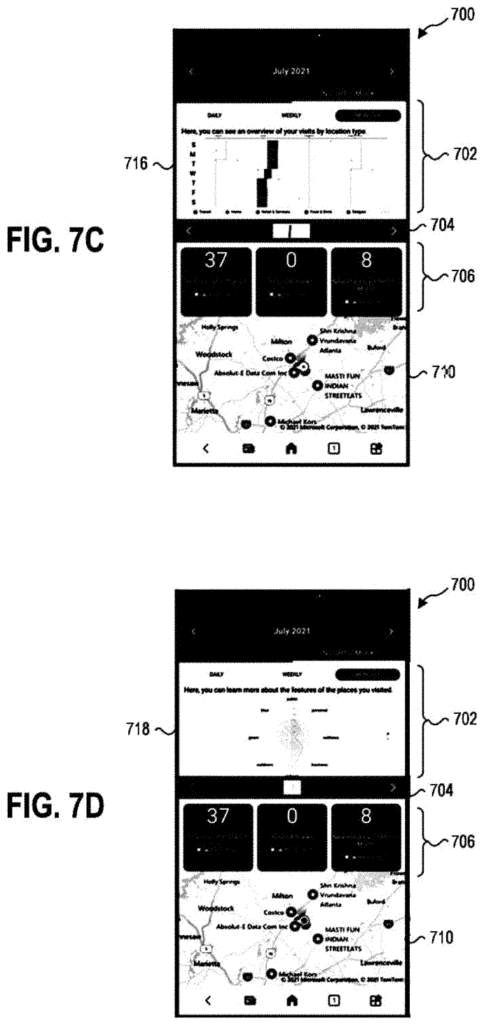

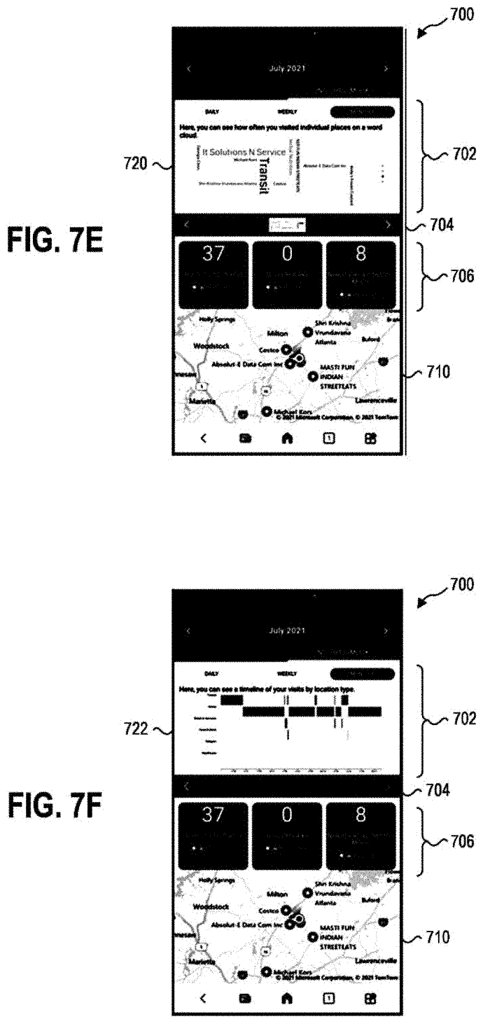

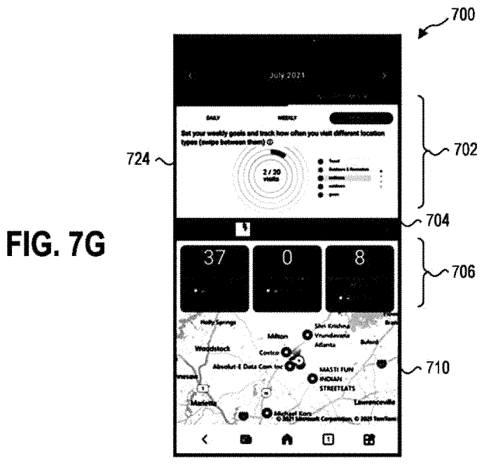

Referring now to FIGS. 7A-7G, illustrated is an example graphical user interface (GUI) screen 700 of the insight dashboard 14 (FIG. 1). The GUI screen 700 may be presented on a display 106 of a user's 104 device 102 (FIG. 1). The GUI screen 700 may present one or more location-aware insights 16 (FIG. 1) on the insight dashboard 14 for a selected time interval or date range. In the illustrated example, the selected time interval is the month of July. The user 104 also has the option to select a daily time interval or a weekly time interval on the GUI screen 700. In addition, the user 104 has the option to move forwards in time (e.g., to August) or backwards in time (e.g., June), for example, by selecting the arrows nearby the selected time interval (e.g., July 2021).

The GUI screen 700 may include a map 710 that presents the different locations visited by the user for the selected time interval. The locations visited may be visually distinct on the map 710. For example, the map 710 uses circles or other icons to identify the locations visited by the user 104 during the time interval. In addition, the GUI screen 700 may include one or more interesting facts 706 presented regarding the locations visited by the user 104. The interesting facts 706 may be presented in an overlay over the map, adjacent to the map, next to the map, below the map, and/or above the map. For example, the interesting facts 706 may indicate that the user 104 lost thirty seven hours in transit during the month of July. The interesting facts 706 may also indicate that the user 104 spent zero hours at parks during the month of July. The interesting facts 706 may also indicate that the user visited eight new places in the month of July.

The GUI screen 700 may include a visual carousel 702 that presents a chart or graph (e.g., doughnut chart 712) to display location-aware insights 16 for the visit history 20 of the user 104 for the selected time interval. The visual carousel 702 may be a nested carousel that allows the user 104 to switch between different charts or graphs for presenting the location-aware insights 16. For example, the user 104 scrolls left or right on the thumbnails 704 to have a different chart or graph presented on the visual carousel 702. In addition, the user 104 may select an individual thumbnail 704 to have the associated chart or graph presented on the visual carousel 702. The thumbnails 704 may be presented below the visual carousel 702, next to the visual carousel 702, adjacent to the visual carousel 702, above the visual carousel 702, and/or in an overlay on the visual carousel 702. In addition, the map 710 may be presented below the visual carousel 702, next to the visual carousel 702, adjacent to the visual carousel 702, above the visual carousel 702, and/or in an overlay on the visual carousel 702. Moreover, the configuration of the GUI screen 700 (e.g., the placement of the visual carousel 702, the thumbnails 704, the interesting facts 706, and/or the map 710 on the GUI screen 700) may be based on the user preferences and/or the display characteristics of the device 102.

FIGS. 7A-7G illustrate the user 104 switching between different charts or graphs to display different location-aware insights 16 for the visit history 20 on the visual carousel 702. FIG. 7A illustrates a doughnut chart 712 providing information on the different location types (e.g., home, food and drink, healthcare, retail) that the user 104 visited during the month of July. FIG. 7B illustrates a graph 714 providing information on the different location types (e.g., home, food and drink, healthcare, retail) that the user 104 visited during the month of July) by the day of the week. For example, the user 104 selected a thumbnail 704 for the graph 714 to display the graph 714 instead of the doughnut chart 712. FIG. 7C illustrates a graph 716 providing information on the different location types (e.g., home, food and drink, healthcare, retail) that the user 104 visited during the month of July by the day of the week. For example, the user 104 scrolled to left or right on the thumbnails 704 using the arrows to the graph 716 and the graph 716 is displayed based on the user 104 scrolling.

FIG. 7D illustrates a radar chart 718 presenting different features of the places visited by the user 104 during the selected date interval. For example, the user 104 may swipe the GUI screen 700 horizontally moving the thumbnails 704 to the radar chart 718 to select the radar chart 718 for display on the visual carousel 702. FIG. 7E illustrates a word cloud 720 showing how often the user 104 visited individual places during the selected time interval. FIG. 7F illustrates a chart 722 with a timeline of the user's 104 visits by location type during the selected time interval. FIG. 7G illustrates a chart 724 tracking the user's 104 weekly goals and how often the user 104 visited different location types during the selected time interval. For example, the user 104 set one or more location-relevant goals and the chart 724 helps the user 104 track the progress for the location-relevant goals.

As the user 104 selects different charts or graphs (e.g., the doughnut chart 712, the graph 714, the graph 716, the radar chart 718, the word cloud 720, the chart 722, or the chart 724) for presentation on the visual carousel 702, the remaining information on the GUI screen 700 may remain the same (e.g., the places visited highlighted on the map 710 and the interesting facts 706). In addition, as the user 104 selects different charts or graphs for display on the visual carousel 702, the remaining information on the GUI screen 700 may change (e.g., different interesting facts 706 may be displayed) and/or different information may be presented on the map 710 (e.g., an animated sequence of the visits may be displayed).

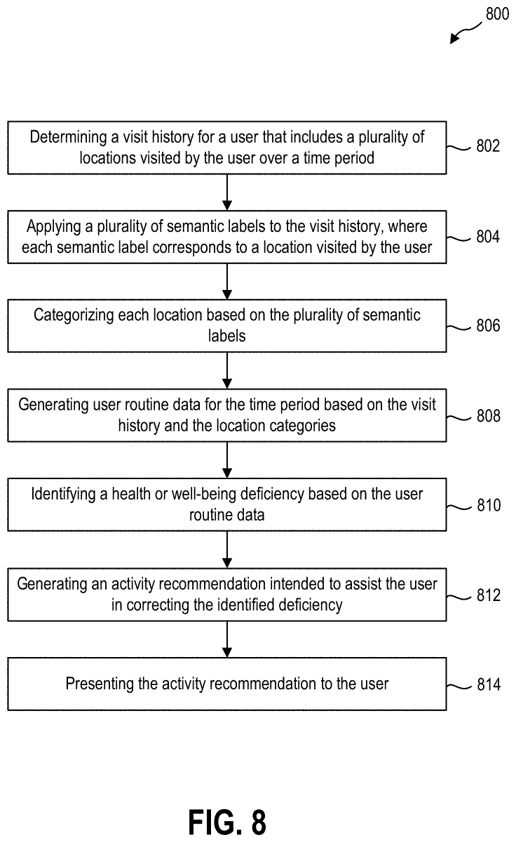

Referring now to FIG. 8, illustrated is an example method 800 for providing location-aware insights 16 (FIG. 1). The actions of the method 800 may be discussed below in reference to the architecture of FIG. 1.

At 802, the method 800 includes determining a visit history for a user that includes a plurality of locations visited by the user over a time period. A visit detection model 108 receives the location data 12 for a user 104 from a device 102 associated with the user 104. The device 102 may include a location tracking component 10 that tracks the location data 12 of the users 104. The location tracking component 10 collects the location data 12 of the user 104 regularly and sends the location data 12 to one or more servers 120 that may host the visit detection model 108. The visit detection model 108 generates a visit history 20 for the location data 12. The visit detection model 108 may filter the location data 12 activity registered by the device 102 to identify and generate a corresponding set of visit histories 20 for the location data 12. The visit detection model 108 may use spatial and temporal features of the location data 12 when generating the visit history 20. The visit history 20 may identify a plurality of locations 21 that the user 104 visited. In addition, the visit history 20 may have an associated date and/or time (start time, end time) for the different locations 21 included in the plurality of locations. As such, the visit history 20 may include a plurality of locations 21 that the user 104 visited over a time period.

The visit histories 20 for the users 104 may be stored in a datastore 112. The datastore 112 may include a visit history database that stores the visit histories 20 by each user 104, and the visit histories 20 may be sorted by date and/or time.

At 804, the method 800 includes applying a plurality of semantic labels to the visit history, where each semantic label corresponds to a location 21 visited by the user. The semantic enrichment component 110 may classify the locations 21 of each visit history 20 and may enrich each visit history 20 with a corresponding semantic label 22. The semantic label 22 may include a name of a business or place if the location 21 is for a public location or may include a personal name (e.g., home or work) created by the user 104. The semantic label 22 may also include a location category (e.g., park, restaurant, office, etc.) for each location 21 included in the visit history 20.

At 806, the method 800 includes, categorizing each location based on the plurality of semantic labels. The semantic enrichment component 110 may categorize each location 21 visited by the user 104 in the visit history 20 with a corresponding location category. The semantic enrichment component 110 may use location graphs to produce the corresponding location categories for the locations 21. In addition, the semantic enrichment component 110 may use other domain graphs that may be used as a complement of the location graph to provide additional information for the locations 21. Example domain graphs includes transportation modes graphs, common sense graphs that describe the concept of time to provide a temporal aspect of the visits, and/or activity graphs that relate locations with physical activities. As such, the semantic label 22 may use the location graphs and/or the domain graphs to assign each location 21 included in the visit history 20 a location name and a location type or category.

The semantic enrichment component 110 may also use the location graphs and/or the domain graphs to identify location attributes for the locations 21 of the visit history 20. The semantic label 22 may include location attributes with adjectives or details describing the locations of the visit history 20. The location attributes may include environmental attributes, such as, but not limited to, air quality, light conditions, noise, indoor spaces, outdoor spaces, open spaces, closed spaces, green spaces, grey spaces, popular spaces, less popular spaces, bright spaces, dark spaces, new spaces, and/or old spaces. The location attributes may also include cost relevant attributes for the locations 21 of the visit history 20.

The semantic enrichment component 110 may enrich the visit histories 20 with a plurality of semantic labels 22 for each location 21 included in the visit histories 20. The semantic labels 22 provide more comprehensive information of the locations included in the visit histories 20. The semantic enrichment component 110 may add the semantic labels 22 to the visit histories 20 stored in the datastore 112.

At 808, the method 800 includes generating user routine data for the time period based on the visit history and the location categories. An analytics component 114 may analyze the visit histories 20 and the semantic labels 22 to identify user routine data 24 with visit patterns that may impact the user's 104 emotional well-being or health. The analytics component 114 may determine location related statistics 24 about the visit histories 20 using the semantic labels 22. The location related statistics 24 may include, but are not limited to, a frequency of visits to location types, a duration of the visits, a regularity of the visits, variety of the visits, location attributes, sequencing/association, and/or a popularity of a location. The analytics component 114 may generate the user routine data 27 based on the location related statistics 24. As such, the user routine data 27 may include a frequency of visits to location types, a duration of the visits, a regularity of the visits, variety of the visits, location attributes, sequencing/association, and/or a popularity of a location. The user routine data 27 may also include transportation information or transportation data for how the user travelled between the plurality of locations 21 included in the user routine data 27.

At 810, the method 800 includes identifying a health or well-being deficiency based on the user routine data. The analytics component 114 may analyze the user routine data 27 and identify any health or well-being deficiencies of the user 104 that may be indicated based on the user routine data 27. The analytics component 114 may focus on the location related statistics 24 of the user routine data 27 that may impact the well-being or health of the user 104 in identifying the health or well-being deficiency, such as, but not limited to, a frequency of visits to location types, a duration of the visits, a regularity of the visits, variety of the visits, location attributes, sequencing/association, and/or a popularity of a location. Other factors of the location and/or the user routine data 24 may be identified by the analytics component 114 as impacting the well-being or health of the user 104. For example, the identified health or well-being deficiency is identified based on a goal set by the user. Another example includes the identified health or well-being deficiency is identified based on the physical health of the user (e.g., blood pressure, heart rates, etc.). Another example includes the identified health or well-being deficiency is identified based on a financial goal of the user 104.

The analytics component 114 may also analyze the user routine data 27 and the semantic labels 22 to generate any predictions 26 about the user 104. The analytics component 114 may estimate the user's 104 future visit patterns based on the analysis of the user routine data 27 and the semantic labels 22. The predictions 26 may provide the users 104 with a possibility to adapt in advance potential negative visit patterns (e.g., to change a visit pattern to reduce an amount of time in traffic based on a high prediction 26 that a long commute time will occur today). In an implementation, a future schedule of the user is predicted by the analytics component 114 by inputting the user routine data 27 into a Markov model. The activity recommendations 28 may be a recommendation to edit or change the predicted future schedule.

The analytics component 114 may operate at multiple semantic levels by covering the individual location instances included in the visit histories 20 and processing the location instances semantic types to provide more insightful insights into the users' 104 movement and visit patterns in the visit histories 20.

At 812, the method 800 includes generating an activity recommendation intended to assist the user in correcting the identified deficiency. The insight component 116 may aggregate the statistics 24 and/or the predictions 26 for the user routine data 27 of the user 104 and may generate one or more location-aware insights 16 based on the statistics 24 and/or the predictions 26. The insight component 116 may use the location related statistics 24 and/or the predictions 26 to identify the user routine data 27 (e.g., the visit and location patterns) that are related to the well-being or health of the user 104. The insight component 116 may also use the location related statistics 24 and/or the predictions 26 to identify or infer the actual level of well-being or health of the user 104. As such, the one or more location-aware insights 16 may relate to a health or well-being of the user 104.

The location-aware insights 16 may highlight visit patterns, statistics 24, and/or predictions 26 of the user 104 to different locations 21 that may affect the well-being or health of the user 104. The location-aware insights 16 may highlight or summarize the user routine data 27 of the user 104 in a relatable manner so that the user 104 may easily identify different location related statistics 24 for the different locations 21 and/or location types related to the well-being or health of the user 104.

The location-aware insights 16 may also include one or more activity recommendations 28 to improve the well-being or health of the user 104. The activity recommendations 28 may provide proactive recommendations for a healthier, location-aware way of living. Example activity recommendations 28 include, booking in advance a free day in the park, motivating the user 104 to change a visit pattern, trying a new activity, and/or visiting a new or different location. The activity recommendations 28 may be tailored to the user 104. For example, the activity recommendations 28 suggest that the user 104 spend more time outside and recommend free time in the user's schedule to visit a nearby park. One example use case is the activity recommendation 28 coordinating with a calendar application to provide recommendations to the user 104 for when to take a break, along with providing recommendations for where to take the break. The activity recommendations 28 may provide recommendations for both when and where to take the break. As such, the activity recommendations 28 may assist the user 104 in correcting any identified health or well-being deficiencies in the user routine data 27.

At 814, the method 800 includes presenting the activity recommendation to the user. The insight component 116 may output the location-aware insights 16 on a user interface of a display 106 of the device 102. The user interface may display an insight dashboard 14 with the location-aware insights 16. The insight dashboard 14 may be a web or native application dashboard. The insight dashboard 14 may be customized to preferences of the user 104. The user 104 may select different charts for viewing the same location-aware insights 16. The location-aware insights 16 may include the user routine data 27, the activity recommendations 28, the statistics 24 related to the user routine data 27, and any predictions 26 for the user routine data 27.

The insight dashboard 14 may present the location-aware insights 16 in a relatable manner so that the user 104 may easily identify or understand the user routine data 27, the activity recommendations 28, the location related statistics 24, and/or other factors of the user routine data 27 that may affect the user's 104 well-being or health. The insight dashboard 14 may use a variety of visuals or modalities for presenting the location-aware insights 16. Examples include graphics, animations, charts, text, speech, reports, and/or push notifications. The insight dashboard 14 may represent different types of information for the location-aware insights 16 using different modalities. For example, the statistics 24 may be presented using graphics or charts and interesting facts may be presented using text.

The insight dashboard 14 allows user interactivity and supports gestures by the users 104 (e.g., pinching zooming, touching, dragging, scrolling, and/or swiping). A date range picker provided by the insight dashboard 14 allows the users 104 to select either individual dates or a date range for generating the location-aware insights 16. The date range may be a set of predefined time intervals (e.g., one week, one month, three months, six months, one year) to help the users 104 quickly identify the location-aware insights 16 that may affect the well-being of the user 104 during the time intervals.

A user 104 may change the selected date and/or the time intervals by a variety of input methods. For example, a time axis slider allows the users 104 to slide back and forth in time (e.g., move back a month or move towards the present time). Another example includes the user 104 selecting arrows to move forwards or backwards in time. As the user moves back and forth in time, a map 18 may interactively update with different locations that the user visited during the selected time interval. As such, the users 104 may easily view the changes in the location visits on the map 18 over a time interval by moving backwards or forwards in time.

The insight dashboard 14 may allow the users 104 to save and tag a group of visits within a certain date range or selected time interval. The insight dashboard 14 may receive user input identifying a portion of the user routine data 27 (e.g., a group of visits to different locations 21) as corresponding to a life event (e.g., vacations or school). The insight dashboard 14 may allow the users 104 to label and/or store the portion of the user routine data 27 with the life event. For example, the users 104 save and tag a long weekend trip to the Rocky Mountains as “My trip to the Rockies.” The users 104 may use the insight dashboard 14 to find and relive/replay the trip to the Rocky Mountains on the insight dashboard 14. That is, in combination with the temporal slider, the insight dashboard 14 has a play and/or pause button and a speed parameter. The users 104 may search for saved and/or tagged visit sequences (e.g., using a drop down menu or text entry) and by pressing the play button, the users 104 may see the corresponding location-aware insights 16 during the journey, as well as watch an animated sequence of visit locations displayed on the map 18 in the order the visits took place, visit by visit, day by day until the end of the trip. In addition, the insight dashboard 14 may coordinate with a camera application and/or a photograph application and display photographs taken at the different visit locations by the user 104. As such, the user 104 may use the insight dashboard 14 to revisit visit histories 20 by saving a visit history 20, tagging the visit history 20 (e.g., with a name for the visit history 20), and/or reliving the visit history 20 (e.g., by viewing an animated sequence of the visit locations displayed on the map 18, the different location-aware insights 16 for the visit history 20, and/or any photographs taken at the visit locations).

In addition, the user 104 may view their visits on a map 18 and filter the visits by date and/or location type. For example, the user 104 filters their locations 21 visited to display all shopping locations visited in the last 2 weeks. An overlay may be displayed nearby each location on the map with information relevant to the visit(s) that took place at the specific location (e.g., location name(s), visit date(s), duration(s), start time(s), end time(s), popularity, location attributes, and/or other statistics 24 for the visit). The overlay may appear when the user 104 selects the location on the map 18.

The insight dashboard 14 may also provide rewards and/or incentives to the users 104 for following the activity recommendations 28 or suggestions from the location-aware insights 16. In addition, the insight dashboard 14 may provide rewards and/or incentives to the users 104 for achieving goals set for visits.

The insight dashboard 14 may also provide shared location-aware insights 16 for a group of users 104 (e.g., family or friends). The activity recommendation 28 included in the shared location-aware insights 16 may be based on aggregate statistics 24 from the group of users 104. For example, parents receive location-aware insights about their children (e.g., an amount of time the children spent outdoors, at school, at entertainment venues, at friend's houses). A family may have a shared insight dashboard 14 that tracks common movement and/or visit patterns of the entire family. The location-aware insights 16 may be used to set goals (e.g., spend more time outside) for the family and the insight dashboard 14 may track the progress towards the goals for each of the family members. The insight dashboard 14 may also be used to create competitions between the family members for achieving the goals and/or providing rewards for achieving the goals.

As such, the method 800 may provide relatable location-aware insights 16 to the user 104 that identifies user routine data 27 (e.g., visit patterns) and/or statistics 24 in the visit histories 20 related to the user's 104 well-being or health. The method 800 may allow the users 104 to gain a better location-aware understanding by reflecting on visit and movement behaviors to help the users 104 find balance and a well-being state.

As illustrated in the foregoing discussion, the present disclosure utilizes a variety of terms to describe features and advantages of the model evaluation system. Additional detail is now provided regarding the meaning of such terms. For example, as used herein, a “machine learning model” refers to a computer algorithm or model (e.g., a classification model, a regression model, a language model, an object detection model) that can be tuned (e.g., trained) based on training input to approximate unknown functions. For example, a machine learning model may refer to a neural network (e.g., a convolutional neural network (CNN), deep neural network (DNN), recurrent neural network (RNN)), or other machine learning algorithm or architecture that learns and approximates complex functions and generates outputs based on a plurality of inputs provided to the machine learning model. As used herein, a “machine learning system” may refer to one or multiple machine learning models that cooperatively generate one or more outputs based on corresponding inputs. For example, a machine learning system may refer to any system architecture having multiple discrete machine learning components that consider different kinds of information or inputs.

The techniques described herein may be implemented in hardware, software, firmware, or any combination thereof, unless specifically described as being implemented in a specific manner. Any features described as modules, components, or the like may also be implemented together in an integrated logic device or separately as discrete but interoperable logic devices. If implemented in software, the techniques may be realized at least in part by a non-transitory processor-readable storage medium comprising instructions that, when executed by at least one processor, perform one or more of the methods described herein. The instructions may be organized into routines, programs, objects, components, data structures, etc., which may perform particular tasks and/or implement particular data types, and which may be combined or distributed as desired in various implementations.

Computer-readable mediums may be any available media that can be accessed by a general purpose or special purpose computer system. Computer-readable mediums that store computer-executable instructions are non-transitory computer-readable storage media (devices). Computer-readable mediums that carry computer-executable instructions are transmission media. Thus, by way of example, and not limitation, implementations of the disclosure can comprise at least two distinctly different kinds of computer-readable mediums: non-transitory computer-readable storage media (devices) and transmission media.

As used herein, non-transitory computer-readable storage mediums (devices) may include RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

The steps and/or actions of the methods described herein may be interchanged with one another without departing from the scope of the claims. In other words, unless a specific order of steps or actions is required for proper operation of the method that is being described, the order and/or use of specific steps and/or actions may be modified without departing from the scope of the claims.

The term “determining” encompasses a wide variety of actions and, therefore, “determining” can include calculating, computing, processing, deriving, investigating, looking up (e.g., looking up in a table, a database, a datastore, or another data structure), ascertaining and the like. Also, “determining” can include receiving (e.g., receiving information), accessing (e.g., accessing data in a memory) and the like. Also, “determining” can include resolving, selecting, choosing, establishing and the like.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional implementations that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by implementations of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to implementations disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the implementations that falls within the meaning and scope of the claims is to be embraced by the claims.

### INDUSTRIAL APPLICABILITY

The present disclosure is related to methods and systems for providing location-aware insights. The location-aware insights highlight or summarize the visit histories of the user in a relatable manner so that the user may easily identify different location related statistics for the different locations and/or location types related to the well-being or health of the user. In some implementations, the location-aware insights include one or more location related activity recommendations to improve the well-being or health of the user. The activity recommendations provide proactive recommendations for a healthier, location-aware way of living. An example suggestion includes visiting a location at a different time of day to reduce an amount of time in traffic.

The methods and systems analyze the visit histories of the users and use semantic location information in analyzing the different locations visited by the user. The methods and systems identify or highlight user routine data (e.g., visit patterns to different locations in the visit histories) that impact the user's emotional well-being or health. The methods and systems determine location related statistics about the user routine data using semantic labels associated with the visit histories. The methods and systems use the location related statistics to infer the level of well-being or health of the user, a mood of the user, and/or certain personality traits of the user (e.g., extroversion). The location related statistics include, but are not limited to, location and visit frequency, duration, regularity, and/or periodicity. The methods and systems operate at multiple semantic levels by covering the individual location instances included in the visit histories and processing the location instances semantic types to provide more insightful information into the users' movement and visit patterns in the visit histories.

The methods and systems generate one or more location-aware insights based on the analysis. The one or more location aware insights may relate to a health or well-being of the user. The methods and systems create a personalized location-aware well-being insight dashboard for users.

The insight dashboard presents the location-aware insights in a relatable manner so that the user may easily identify or understand the location related statistics and/or other factors of the user routine data that may affect the user's well-being or health. The insight dashboard uses a variety of visuals or modalities for presenting the location-aware insights. Examples include graphics, animations, charts, text, speech, reports, and/or push notifications. The insight dashboard supports user interactivity and gestures by the users (e.g., pinching zooming, touching, dragging, scrolling, and/or swiping). The insight dashboard helps users keep track of the time that the users invested during the day, week, month (in a retrospective manner) grouped by location category.

The insight dashboard allows the users to replay visit histories by saving a visit history, tagging the visit history (e.g., with a name for the visit history), and/or reliving the visit history (e.g., by viewing an animated sequence of the visit locations displayed on the map, the different location-aware insights for the visit history, and/or any photographs taken at the visit locations). The insight dashboard also allows users to set location-relevant goals and tracks the progress of the users towards achieving the location-relevant goals. The insight dashboard also provides shared location-aware insights for a group of users (e.g., a family, friends, co-workers, and/or contacts). The shared location-aware insights may track movement or visit patterns for the group of users and provide statics or other relevant information for the movement or visit patterns that may affect the user's well-being or health.

As such, the methods and systems give the user deeper insights about their visits patterns and the corresponding dwelling times at the different locations. The methods and systems allow users to gain a better location-aware understanding by providing location-aware insights related to the user's mental well-being or physical health. The methods and systems help users better understand where and how the users spend their time and helps users understand whether any visit patterns need to change to promote a healthier lifestyle or improve the health or metal well-being of the users.

(A1) Some implementations include a method for providing location-aware insights (e.g., location-aware insights 16). The method includes determining (802), using a visit detection model (e.g., visit detection model 108), a visit history (e.g., visit history 20) for a user (e.g., user 104) that includes a plurality of locations (e.g., locations 21) visited by the user over a time period, where the visit detection model receives location data (e.g., location data 12) from a device (e.g., device 102) of the user and determines the plurality of locations visited based on the location data. The method includes applying (804), by a semantic enrichment component (e.g., semantic enrichment component 110), a plurality of semantic labels (e.g., semantic label 22) to the visit history, where each semantic label in the plurality of semantic labels corresponds to a location in the plurality of locations visited by the user. The method includes categorizing (806) each location in the plurality of locations based on the plurality of semantic labels, where each location category has one or more corresponding environmental attributes. The method includes generating (808), using an analytics component (e.g., analytics component 114), user routine data (e.g., user routine data 27) for the time period based on the visit history and the location categories. The method includes identifying (810) a health or well-being deficiency based on the user routine data. The method includes generating (812), by an insight component (e.g., insight component 116), an activity recommendation (e.g., activity recommendation 28) intended to assist the user in correcting the identified deficiency. The method includes presenting (814) the activity recommendation to the user.

(A2) In some implementations of the method of A1, the activity recommendation is further based on aggregate statistics from a plurality of users.

(A3) In some implementations, the method of A1 or A2 includes receiving user input identifying a portion of the user routine data as corresponding to a life event; and labelling and storing the portion of the user routine with the life event.

(A4) In some implementations of the method of any of A1-A3, a location graph is used to categorize the locations.

(A5) In some implementations of the method of any of A1-A4, the environmental factors include one or more of air quality, light conditions, noise, outdoor spaces, indoor spaces, green spaces, grey spaces, popular spaces, public places, private places, new spaces, or old spaces.

(A6) In some implementations of the method of any of A1-A5, the user routine data includes one or more of a frequency of visits to location types, a duration of a visit to a location, a regularity of visits to a location, a variety of visits to locations, location attributes, or popularity of a location.

(A7) In some implementations of the method of any of A1-A6, the user routine data includes transportation data for how the user travelled between the plurality of locations.

(A8) In some implementations, the method of any of A1-A7 includes predicting a future schedule of the user by inputting the user routine data into a Markov model, where the activity recommendation is a recommendation to edit the predicted future schedule.

(A9) In some implementations of the method of any of A1-A8, the identified deficiency is determined based on a goal set by the user.

(A10) In some implementations of the method of any of A1-A9, the identified deficiency is determined based on physical health of the user.

(A11) In some implementations of the method of any of A1-A10, the identified deficiency is determined based on a financial goal of the user.

(A12) In some implementations of the method of any of A1-A11, the activity recommendation is presented as part of an insight dashboard (e.g., insight dashboard 14) that displays statistics (e.g., statistics 24) for the user routine data and includes a map (e.g., map 18) displaying the plurality of locations visited by the user during the time period.

(B1) Some implementations include a user interface presented on a display (e.g., 106) of a device (102). The user interface includes an insight dashboard (e.g., insight dashboard 14) that displays one or more location-aware insights (e.g., location-aware insights 16) with location related statistics (e.g., statistics 24) for a visit history (e.g., visit history 20) of a user (e.g., user 104) for a date or time interval selected by the user, where the one or more location-aware insights relate to a health or well-being of the user. The insight dashboard includes a map (e.g., map 18) nearby the one or more location-aware insights that displays a plurality of locations visited by the user in the visit history during the date or the time interval.

(B2) In some implementations of the user interface of B 1, when the user selects a different date or a different time interval for the visit history, the map updates the plurality of locations visited by the user in the visit history, and the one or more location-aware insights updates the location related statistics for the different date or the different time interval.

(B3) In some implementations of the user interface of B1 or B2, the insight dashboard includes an overlay on the map that provides information about a visit of the user to each location of the plurality of locations, where the information includes one or more of a start time of the visit, an end time of the visit, a duration of the visit, a location name, location semantics, a popularity score of the location, or location attributes.

(B4) In some implementations of the user interface of any of B1-B3, the insight dashboard includes a visual carousel (e.g., visual carousel 416) displaying the one or more location-aware insights using one or more charts or graphs, where the visual carousel displays a chart or a graph for the one or more location-aware insights based on input from the user.

(B5) In some implementations of the user interface of any of B1-B4, the insight dashboard includes thumbnails (e.g., thumbnails 418, 420, 422, 424, 426) identifying the charts or the graphs for presenting the one or more location-aware insights, and the visual carousel displays the chart or the graph associated with a selected thumbnail, wherein the thumbnail is selected by the user swiping the visual carousel or the user selecting the thumbnail.

Some implementations include a system (environment 100). The system includes one or more processors; memory in electronic communication with the one or more processors; and instructions stored in the memory, the instructions being executable by the one or more processors to perform any of the methods described here (e.g., A1-A12, B1-B5).

Some implementations include a computer-readable storage medium storing instructions executable by one or more processors to perform any of the methods described here (e.g., A1-A12, B1-B5).

The present disclosure may be embodied in other specific forms without departing from its spirit or characteristics. The described implementations are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A method for providing location-aware insights, comprising:
determining a visit history for a user that includes a plurality of locations visited by the user over a time period by using location data received from a device of the user and determining the plurality of locations visited based on the location data;
applying a plurality of semantic labels to the visit history, wherein each semantic label in the plurality of semantic labels corresponds to a location in the plurality of locations visited by the user;
categorizing each location in the plurality of locations based on the plurality of semantic labels, wherein each location category has one or more corresponding environmental attributes;
generating user routine data for the time period based on the visit history and the location categories;
identifying a health or well-being deficiency based on the user routine data;
generating an activity recommendation intended to assist the user in correcting the identified deficiency; and
presenting the activity recommendation to the user.

2. The method of claim 1, wherein the activity recommendation is further based on aggregate statistics from a plurality of users.

3. The method of claim 1, further comprising:
receiving user input identifying a portion of the user routine data as corresponding to a life event; and
labelling and storing the portion of the user routine with the life event.

4. The method of claim 1, wherein a location graph is used to categorize the locations.

5. The method of claim 1, wherein the environmental attributes include one or more of air quality, light conditions, noise, outdoor spaces, indoor spaces, green spaces, grey spaces, popular spaces, public places, private places, new spaces, or old spaces.

6. The method of claim 1, wherein the user routine data includes one or more of a frequency of visits to location types, a duration of a visit to a location, a regularity of visits to a location, a variety of visits to locations, location attributes, or popularity of a location.

7. The method of claim 1, wherein the user routine data includes transportation data for how the user travelled between the plurality of locations.

8. The method of claim 1, further comprising:
predicting a future schedule of the user by inputting the user routine data into a Markov model, wherein the activity recommendation is a recommendation to edit the predicted future schedule.

9. The method of claim 1, wherein the identified deficiency is determined based on a goal set by the user.

10. The method of claim 1, wherein the identified deficiency is determined based on physical health of the user.

11. The method of claim 1, wherein the identified deficiency is determined based on a financial goal of the user.

12. The method of claim 1, wherein presenting the activity recommendation further comprises:
presenting the activity recommendation as part of an insight dashboard that displays statistics for the user routine data and includes a map displaying the plurality of locations visited by the user during the time period.

13. A system, comprising:
one or more processors;
memory in electronic communication with the one or more processors;
a visit detection model, a semantic enrichment component, an analytics component, and an insight component in electronic communication with the one or more processors and the memory; and
instructions stored in the memory, the instructions executable by the one or more processors to cause one or more of the detection model, the semantic enrichment component, the analytics component, or the insight component to:
determine a visit history for a user that includes a plurality of locations visited by the user over a time period by using location data received from a device of the user and determining the plurality of locations visited based on the location data;
apply a plurality of semantic labels to the visit history, wherein each semantic label in the plurality of semantic labels corresponds to a location in the plurality of locations visited by the user;
categorize each location in the plurality of locations based on the plurality of semantic labels, wherein each location category has one or more corresponding environmental attributes;
generate user routine data for the time period based on the visit history and the location categories;
identifying a health or well-being deficiency based on the user routine data;
generate an activity recommendation intended to assist the user in correcting the identified deficiency; and
present the activity recommendation to the user.

14. The system of claim 13, wherein the activity recommendation is further based on aggregate statistics from a plurality of users.

15. The system of claim 13, wherein the instructions are further executable by the one or more processors to cause one or more of the detection model, the semantic enrichment component, the analytics component, or the insight component to:
receive user input identifying a portion of the user routine data as corresponding to a life event; and
label and store the portion of the user routine with the life event.

16. The system of claim 13, wherein a location graph is used to categorize the locations.

17. The system of claim 13, wherein the environmental attributes include one or more of air quality, light conditions, noise, outdoor spaces, indoor spaces, green spaces, grey spaces, popular spaces, public places, private places, new spaces, or old spaces.

18. The system of claim 13, wherein the user routine data includes one or more of a frequency of visits to location types, a duration of a visit to a location, a regularity of visits to a location, a variety of visits to locations, location attributes, or popularity of a location.

19. The system of claim 13, wherein the user routine data includes transportation data for how the user travelled between the plurality of locations.

20. The system of claim 13, wherein the identified deficiency is determined based on one or more of a goal set by the user, physical health of the user, or a financial goal of the user.
